(19) United States
(12) Patent Application Publication
(10) Pub. No.: US 2015/0031765 A1
(43) Pub. Date: Jan. 29, 2015

(54) TREATMENT OF COGNITIVE IMPAIRMENT

(71) Applicants: Maurice Robert CROSS, Guernsey (GB); Mikael S. THOMSEN, Hvidovre (DK); Jenny HAGGLOF, Malmo (SE)

(72) Inventors: Maurice Robert CROSS, Guernsey (GB); Mikael S. THOMSEN, Hvidovre (DK); Jenny HAGGLOF, Malmo (SE)

(21) Appl. No.: 14/513,414

(22) Filed: Oct. 14, 2014

Related U.S. Application Data
(63) Continuation of application No. 14/236,354, now abandoned.

(30) Foreign Application Priority Data
Aug. 2, 2011 (DK) PA 2011 70422

(51) Int. Cl. A61K 31/19

(52) U.S. Cl. CPC A61K 31/19
USPC 514/557; 562/498

(57) ABSTRACT

The present invention relates to a compound with the core structure of formula (I), or pharmaceutically acceptable salts or esters, solvates, or prodrugs thereof, for use in the treatment of cognitive impairment in a mammal. The compound is preferably isosteviol or steviol, or pharmaceutically acceptable salts or esters, solvates, or prodrugs thereof.

### FIELD OF INVENTION

The present invention relates to the use of compounds of formula (I) for the treatment of cognitive impairment.

### BACKGROUND OF INVENTION

Cognitive impairment occurs when problems with thought processes are present. It can include loss of higher reasoning, forgetfulness, learning disabilities, concentration difficulties, decreased intelligence, and other reductions in mental functions. Cognitive impairment may be present at birth or can occur at any point in a person's lifespan.

Subjects with mild cognitive deficiencies are able to function in everyday activities but have difficulty with memory, i.e. trouble remembering the names of people they met recently, remembering the flow of a conversation, and a tendency to misplace things. The subject may be aware of these difficulties and compensate with increased reliance on notes and calendars and other tools/activities. Through further development of the cognitive deficiency memory loss has progressed to such a point that normal independent function is impossible and the individual can no longer successfully provide for their own basic needs.

While Alzheimer's disease of the familial or the sporadic type is the major cognitive deficiency found in the aging population, other types of cognitive deficiencies are also found. These include but are not limited to: the fronto-temporal degeneration associated with Pick's disease, vascular dementia, senile dementia of Lewy body type, dementia of Parkinsonism with frontal atrophy, progressive supranuclear palsy and corticobasal degeneration and Downs syndrome associated Alzheimers'. Plaque formation is also seen in the spongiform encephalopathies such as CJD, scrapie and BSE.

### Alzheimer's Disease (AD)

Alzheimer's disease (AD), also called Alzheimer disease, senile dementia of the Alzheimer type, primary degenerative dementia of the Alzheimer's type, or simply Alzheimer's, is the most common form of dementia. This presently incurable, degenerative, and terminal disease was first described by German psychiatrist and neuropathologist Alois Alzheimer in 1906 and was named after him. Most often, it is diagnosed in people over 65 years of age, although the less-prevalent early-onset Alzheimer's can occur much earlier.

Although the course of Alzheimer's disease is unique for every individual, there are many common symptoms. The earliest observable symptoms are often mistakenly thought to be ‘age-related’ concerns, or manifestations of stress. In the early stages, the most common symptom is inability to acquire new memories, observed as difficulty in recalling recently observed events. When AD is suspected, the diagnosis is usually confirmed with behavioural assessments and cognitive tests, often followed by a brain scan if available.

As the disease advances, symptoms include confusion, irritability and aggression, mood swings, language breakdown, long-term memory loss, and the general withdrawal of the sufferer as their senses decline. Gradually, bodily functions are lost, ultimately leading to death. Individual prognosis is difficult to assess, as the duration of the disease varies. AD develops for an indeterminate period of time before becoming fully apparent, and it can progress undiagnosed for years. The mean life expectancy following diagnosis is approximately seven years. Fewer than three percent of individuals live more than fourteen years after diagnosis.

The cause and progression of Alzheimer's disease are presently not well understood. Research indicates that the disease is associated with plaques and tangles in the brain. Currently used treatments offer a small symptomatic benefit; no treatments to delay or halt the progression of the disease are, as of yet, available. A number of non-invasive, life-style habits have been suggested for the prevention of Alzheimer's disease, but there is a lack of adequate evidence for a link between these recommendations and reduced degeneration. Mental stimulation, exercise, and a balanced diet are suggested, as both a possible prevention and a sensible way of managing the disease.

The disease course is divided into four stages, with progressive patterns of cognitive and functional impairments.

Pre-dementia: The first symptoms are often mistakenly attributed to aging or stress. Detailed neuropsychological testing can reveal mild cognitive difficulties up to eight years before a person fulfils the clinical criteria for diagnosis of AD. These early symptoms can affect the most complex daily living activities. The most noticeable deficit is memory loss, which shows up as difficulty in remembering recently learned facts and inability to acquire new information. Subtle problems with executing ?? functions of attentiveness, planning, flexibility, and abstract thinking, or impairments in semantic memory (memory of meanings, and concept relationships) can also be symptomatic of the early stages of AD. Apathy can be observed at this stage, and remains the most persistent neuropsychiatric symptom throughout the course of the disease. The preclinical stage of the disease has also been termed mild cognitive impairment, but whether this term corresponds to a different diagnostic stage or identifies the first step of AD is a matter of dispute.

Early stage: In people with AD the increasing impairment of learning and memory eventually leads to a definitive diagnosis. In a small portion of them, difficulties with language, executive functions, perception (agnosia), or execution of movements (apraxia) are more prominent than memory problems. AD does not affect all memory capacities equally. Older memories of the person's life (episodic memory), facts learned (semantic memory), and implicit memory (the memory of the body on how to do things, such as using a fork to eat) are affected to a lesser degree than new facts or memories. Language problems are mainly characterised by a shrinking vocabulary and decreased word fluency, which lead to a general impoverishment of oral and written language. In this stage, the person with Alzheimer's is usually capable of adequately communicating basic ideas. While performing fine motor tasks such as writing, drawing or dressing, certain movement coordination and planning difficulties (apraxia) may be present but they are commonly unnoticed. As the disease progresses, people with AD can often continue to perform many tasks independently, but may need assistance or supervision with the most cognitively demanding activities.

Moderate stage: Progressive deterioration eventually hinders independence; with subjects being unable to perform most common activities of daily living. Speech difficulties become evident due to an inability to recall vocabulary, which leads to frequent incorrect word substitutions (paraphasias). Reading and writing skills are also progressively lost. Complex motor sequences become less coordinated as time passes and AD progresses, so the risk of falling increases. During this phase, memory problems worsen, and the person may fail to recognise close relatives. Long-term memory, which was previously intact, becomes impaired. Behavioural and neuropsychiatric changes become more prevalent. Common manifestations are wandering, irritability and labile affect, leading to crying, outbursts of unpremeditated aggression, or resistance to caregiving. Sundowning can also appear. Approximately 30% of patients develop illusionary misidentifications and other delusional symptoms. Subjects also lose insight of their disease process and limitations (anosognosia). Urinary incontinence can develop. These symptoms create stress for relatives and caretakers, which can be reduced by moving the person from home care to other long-term care facilities.

Advanced stage: During this last stage of AD, the patient is completely dependent upon caregivers. Language is reduced to simple phrases or even single words, eventually leading to complete loss of speech. Despite the loss of verbal language abilities, patients can often understand and return emotional signals. Although aggressiveness can still be present, extreme apathy and exhaustion are much more common results. Patients will ultimately not be able to perform even the simplest tasks without assistance. Muscle mass and mobility deteriorate to the point where they are bedridden, and they lose the ability to feed themselves. AD is a terminal illness, with the cause of death typically being an external factor, such as infection of pressure ulcers or pneumonia, not the disease itself.

Dementia with Lewy Bodies (DLB)

Dementia with Lewy bodies (DLB), also known under a variety of other names including Lewy body dementia, diffuse Lewy body disease, cortical Lewy body disease, and senile dementia of Lewy type, is a type of dementia closely allied to both Alzheimer's and Parkinson's Diseases. It is characterized anatomically by the presence of Lewy bodies, clumps of alpha-synuclein and ubiquitin protein in neurons, detectable in post-mortem brain biopsies.

Dementia with Lewy bodies overlaps clinically with Alzheimer's disease and Parkinson's disease, but is more associated with the latter. With DLB, the loss of cholinergic (acetylcholine-producing) neurons is thought to account for the degradation of cognitive functioning, as in Alzheimer's disease; while the loss of dopaminergic (dopamine-producing) neurons is thought to account for the degradation of motor control, as in Parkinson's disease. Thus, DLB is similar in some ways to both the dementia resulting from Alzheimer's disease and the movement problems of Parkinson's disease. The overlap of neuropathologies and presenting symptoms (cognitive, emotional, and motor) can make an accurate differential diagnosis difficult. In fact, it is often confused in its early stages with Alzheimer's disease and/or vascular dementia (multi-infarct dementia) although, where Alzheimer's disease usually begins quite gradually, DLB often has a rapid or acute onset, with especially rapid decline in the first few months. DLB tends to progress more quickly than Alzheimer's disease.

DLB is distinguished from the dementia that sometimes occurs in Parkinson's Disease by the time frame in which dementia symptoms appear relative to Parkinson symptoms. Parkinson's disease with dementia (PDD) would be the diagnosis when dementia onset is more than 1 year after the onset of Parkinson's. DLB is diagnosed when cognitive symptoms begin at the same time or within a year of Parkinson symptoms.

Pathologically, DLB is characterized by the development of abnormal proteinaceous (alpha-synuclein) cytoplasmic inclusions, called Lewy bodies, throughout the brain. These inclusions have similar structural features to “classical” Lewy bodies seen subcortically in Parkinson's disease. Additionally, there is a loss of dopamine-producing neurons (in the substantia nigra) similar to that seen in Parkinson's disease, and a loss of acetylcholine-producing neurons (in the basal nucleus of Meynert and elsewhere) similar to that seen in Alzheimer's disease. Cerebral atrophy (or shrinkage) also occurs as the cerebral cortex degenerates. Autopsy series have revealed that the pathology of DLB is often concomitant with the pathology of Alzheimer's disease. That is, when Lewy body inclusions are found in the cortex, they often co-occur with Alzheimer's disease pathology found primarily in the hippocampus, including: senile plaques (deposited beta-amyloid protein), and granulovacuolar degeneration (grainy deposits within, and a clear zone around hippocampal neurons). Neurofibrillary tangles (abnormally phosphorylated tau protein) are less common in DLB, although they are known to occur. It is presently not clear whether DLB is an Alzheimer's variant or a separate disease entity.

### Parkinson's Disease Dementia (PDD)

Dementia is a less common feature of Parkinson's disease. Approximately 20% of people with Parkinson's disease will develop Parkinson's Disease Dementia (PDD). Parkinson's patients who experience hallucinations and more severe motor control problems are at risk for dementia. For those patients with Parkinson's disease who go on to develop dementia, there is usually at least a 10- to 15-year lag time between their Parkinson's diagnosis and the onset of dementia.

Parkinson's Disease Dementia (PDD) is different from a similar disorder, known as Dementia with Lewy Bodies (DLB). DLB is characterized by fluctuations in alertness and attention, recurrent visual hallucinations, and parkinsonian motor symptoms like rigidity and the loss of spontaneous movement. In this disorder, the cognitive problems, such as hallucinations, tend to occur much earlier in the course of the disease and often precede the difficulties with walking and motor control.

Indications that dementia may be caused by something other than Parkinson's disease include agitation, delusions (strongly held false beliefs), language difficulties, and early onset of memory symptoms.

Alzheimer's disease and Parkinson's disease are both common in the elderly, especially in those over 85. Therefore, patients with Parkinson's who develop dementia may develop Alzheimer's dementia as well.

The main difference between Parkinson's Disease dementia and Lewy Body dementia is not completely well defined. If motor symptoms come first, by at least a year, and dementia develops later, the convention is to call it Parkinson's Disease dementia. If the motor symptoms follow the dementia symptoms, the convention is to call it Lewy Body dementia (or dementia with Lewy bodies”).

### APOE Gene

Apolipoprotein E (APOE) is a class of apolipoprotein found in the chylomicron and IDLs that binds to a specific receptor on liver cells and peripheral cells.

APOE is essential for the normal catabolism of triglyceride-rich lipoprotein constituents. APOE was initially recognized for its importance in lipoprotein metabolism and cardiovascular disease. More recently, it has been studied for its role in several biological processes not directly related to lipoprotein transport, including Alzheimer's disease (AD), immunoregulation, and cognition.

There are at least three slightly different versions (alleles) of the APOE gene. The major alleles are called e2, e3, and e4. The most common allele is e3, which is found in more than half of the general population. These allelic forms differ from each other only by amino acid substitutions at positions 112 and 158.

The e4 variant is the largest known genetic risk factor for late-onset sporadic Alzheimer's Disease (AD) in a variety of ethnic groups. Caucasian and Japanese carriers of 2 e4 alleles have between 10 and 30 times the risk of developing AD by 75 years of age, as compared to those not carrying any e4 alleles. While the exact mechanism of how e4 causes such dramatic effects remains to be fully determined, evidence has been presented suggesting an interaction with amyloid. Alzheimer's Disease is characterized by build-ups of aggregates of the peptide beta-amyloid. Apolipoprotein E enhances proteolytic break-down of this peptide, both within and between cells. Some isoforms of ApoE are not as efficient as others at catalyzing these reactions. In particular, the isoform ApoE-e4 is not very effective, resulting in increased vulnerability to Alzheimer's in individuals with that gene variation.

Although 40-65% of AD patients have at least one copy of the 4 allele, ApoE-e4 is not a determinant of the disease—at least a third of patients with AD are ApoE-e4 negative and some ApoE-e4 homozygotes never develop the disease. Yet those with two e4 alleles have up to 20 times the risk of developing AD. There is also evidence that the ApoE-e2 allele may serve a protective role in AD. Thus, the genotype most at risk for Alzheimer's disease and at earlier age is ApoE 4,4. The ApoE 3,4 genotype is at increased risk, though not to the degree that those homozygous for ApoE-e4 are. The genotype ApoE 3,3 is considered at normal risk for Alzheimer's disease. The genotype ApoE 2,3 is considered at less risk for Alzheimer's disease. Interestingly, people with both a copy of the 2 allele and the 4 allele, ApoE 2,4, are at normal risk similar to the ApoE 3,3 genotype.

Cognitive deficiencies may also be caused by traumas, such as traumas to the head. The cognitive deficiencies may arise immediately after the trauma or may develop at a later stage.

Regardless on how the cognitive deficiency has developed there is a need to investigate how this deficiency can be treated. Hence, during time a number of studies have been performed in order to investigate target compounds effect on cognitive deficiencies.

WO 2009/071277 is directed to novel nutraceutical compositions containing Stevia extracts or its constituents, such as steviol and stevioside, which may be useful for improvement of cognitive functions, such as learning, memory and alertness and psychotic stability. Hence, the document teaches that beneficial effects in form of improved cognitive functions can be obtained by administering Stevia extract to healthy persons. The document is silent with regard to any beneficial effect on people that are already suffering from cognitive impairment. Neither does the document suggest that Stevia extract may have the same beneficial effect on a sick person as on a healthy person.

WO 2008/134828 relates to diterpenoic compounds, such as isosteviol and steviol, and the diterpene-O-tetrahydro-pyran derivatives, such as for example rebaudioside A, rebaudioside B and rebaudioside C, for use in the treatment of a PPAR deficiency disorder or to the use of these compounds to increase the expression of the peroxisome proliferator activated receptor (PPAR) genes and/or activity of peroxixome proliferator activated receptor (PPAR) and to treat PPAR deficiency-related disorders. In particular it is disclosed that these compounds can be used in the treatment of PPAR activation to prevent the occurrence or to retard the progress of age-related macular degeneration (AMD) or Alzheimer disease (AD). In the experimental part of the application, Example 4 discloses stevioside and steviol's effect on arthrosclerosis, and Example 5 investigate stevioside, rebaudioside and steviol's effect on triglyceride accumulation in adipocytes. The document does not contain any experiments, where the effect on Alzheimer's disease has been investigated.

More recently a study has been published, in which the antiamnesic effect of stevioside has been investigated (Sharma et al., “Antiamnesic effect of stevioside in scopolamine-treated rats”, Indian J. Pharmacol., 2010; 42(3), 164-167). In this study the potential of stevioside in memory dysfunction of rats was investigated by subjecting scopolamine-treated rats to the Morris water maze test. The study concludes that stevioside exerts a memory-preservative effect in cognitive deficits of rats, and explains this beneficial effect by virtue of stevioside's sweetening, antioxidative, anti-inflammatory and anticholinesterase actions. The conclusion that it is the sweetening effect of stevioside that is important for showing the beneficial effect is explained by summarising studies in the recent past that have documented the important role of sweetening agent, i.e. glucose, in learning and memory, and there is extensive evidence in the literature indicating that modest increase in circulating glucose levels enhances the formation of new memories in rodents and humans. Glucose administration has been reported to enhance the memory processes by increasing hippocampal Ach synthesis and release. Furthermore, extracellular brain glucose levels have been demonstrated to vary with neuronal activity, suggesting that circulating glucose may be critical in modulating neural processes important for memory functioning.

### SUMMARY OF INVENTION

The present application addresses the problem of cognitive impairment and cognitive deficiencies, wherein the individual has lost some of the cognitive skills due to a disease or trauma. Particularly the invention relates to treatment of deficiencies where neurons are subject to neurotoxic effects of pathogenic agents such as protein plaques as well as cognitive deficiencies that are caused by physical traumatic exposure to the head, especially from violent sports activities such as boxing, rugby, ice hockey etc., including those cognitive deficiencies that do not show until later in life. Furthermore, the present invention provides compositions for use in treating these and the aforesaid disorders, wherein the individual has lost some of the cognitive skills due to a disease or trauma.

Accordingly, the present invention relates to a compound with the core structure of formula (I), or pharmaceutically acceptable salts or esters, solvates, or prodrugs thereof, for the treatment of cognitive impairment and/or alleviating symptoms of cognitive deficiencies.

The compound is preferably isosteviol or steviol, or pharmaceutically acceptable salts or esters, solvates, or prodrugs thereof, with the proviso that the active compound is not stevioside, steviolbioside, rebaudioside A, rebaudioside B or rebaudioside C.

### DETAILED DESCRIPTION OF THE INVENTION

The present invention relates to treatment and/or alleviating of cognitive impairment and of symptoms of cognitive deficiencies. In particularly the invention relates to treatment of cognitive impairment wherein the deficiencies are caused by neurotoxic effects of pathogenic agents, such as protein plaques. Accordingly, the present invention relates to treatment of cognitive impairment due to an underlying disease. Therefore, in one embodiment the invention relates to treatment of cognitive impairment caused by a cognitive impairment condition, where the cognitive impairment condition is selected from the group consisting of Alzheimer's, Dementia with Lewy bodies and Parkinson's disease dementia. More specifically the invention relates to treatment of cognitive impairment caused by a cognitive impairment condition, where the cognitive impairment condition is Alzheimer's in the early to moderate stage.

The diagnosis of Alzheimers Disease is essentially defined as being present in human patients. However, Alzheimers-like disease has been identified in all common mammal species including the horse, dog and cat. The disease has been induced in animal models such as the mouse so it is likely that some variant of the disease may be present ubiquitously throughout the mammal species. The treatment therefore relates to treatment of cognitive impairment in various mammals, in particularly in human beings, dogs, cats, and horses, most preferably in human beings.

Accordingly, the present invention relates to compounds with the core structure of formula (I)

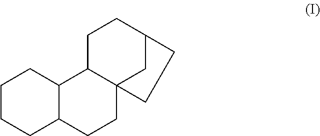 [SMILES: C1CCC2C(C1)CCC13CCC(CCC21)C3]

wherein the core structure is substituted with one or more substituents, or pharmaceutically acceptable salts or esters, solvates, or prodrugs thereof for use in the treatment of cognitive impairment by administering an effective therapeutic amount of said active compound to a mammal subject,
with the proviso that the active compound is not stevioside, steviolbioside, rebaudioside A, rebaudioside B or rebaudioside C. In one embodiment the compound is a compound selected from the group consisting of: isosteviol and steviol.

In a preferred embodiment of the invention the core structure of formula (I), is a core structure of formula (II)

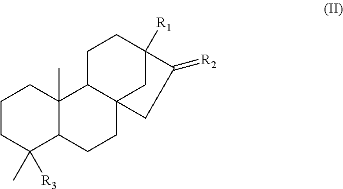

wherein
R₁ is selected from the group consisting of —C₁₋₆alkyl, —O—C₁₋₆alkyl, —OH, and —OC(O)(C₁₋₆alkyl), —COO(C₁₋₆alkyl);
R₂ is selected from the group consisting of CH₂, O, and CH(C₁₋₆alkyl); and
R₃ is selected from the group consisting of —COOH, —COO(C₁₋₆alkyl), —C(O)NH(C₁₋₆alkyl), —C(O)-(common amino acid moiety);
with the proviso that the active compound is not stevioside, steviolbioside, rebaudioside A, rebaudioside B or rebaudioside C.

In a preferred embodiment R₁ is selected from the group consisting of C₁₋₃alkyl, —O—C₁₋₃alkyl, —OH, —OC(O)(C₁₋₃alkyl) and —COO(C₁₋₃alkyl), and in a more preferred embodiment R₁ is selected from the group consisting of methyl, ethyl, —O— methyl, —O-ethyl, OH, —OC(O)CH₃, —OC(O)C₂H₅, —COOCH₃ and —COOC₂H₅, and even more preferred R1 is selected from the group consisting methyl, —O-methyl, —OH, —OC(O)CH₃ and —COOCH₃. Most preferred R1 is selected from the group consisting of methyl and —OH.

In a preferred embodiment R₂ is selected from the group consisting of CH₂, O, and CH(C₁₋₃alkyl), more preferred from the group consisting of CH₂, O, and CH—CH₃, CH—C₂H₅. Most preferred R₂ is selected from the group consisting of CH₂ and O.

In a preferred embodiment R₃ is selected from the group consisting of —COOH, —COO(C₁₋₃alkyl), —C(O)NH(C₁₋₃alkyl), —C(O)-(common amino acid moiety), more preferred from the group consisting of —COOH, —COO(CH₃), —COO(C₂H₅), —C(O)NH(CH₃), —C(O)NH(C₂H₅), and —C(O)-(common amino acid moiety). In the most preferred embodiment R₃ is —COOH.

In one embodiment R₁ is selected from the group consisting of methyl and —OH, R₂ is selected from the group consisting of CH₂, O, and CH—CH₃, CH—C₂H₅, and R₃ is selected from the group consisting of —COOH, —COO(CH₃), —COO(C₂H₅), —C(O)NH(CH₃), —C(O)NH(C₂H₅), and —C(O)-(common amino acid moiety).

In one embodiment R₁ is selected from the group consisting of methyl, —O-methyl, —OH, —OC(O)CH₃ and —COOCH₃, R₂ is selected from the group consisting of CH₂ and O, and R₃ is selected from the group consisting of —COOH, —COO(CH₃), —COO(C₂H₅), —C(O)NH(CH₃), —C(O)NH(C₂H₅), and —C(O)-(common amino acid moiety).

In one embodiment R₁ is selected from the group consisting of methyl, ethyl, —O-methyl, —O-ethyl, OH, —OC(O)CH₃, —OC(O)C₂H₅, —COOCH₃ and —COOC₂H₅, R₂ is selected from the group consisting of CH₂ and O, and R₃ is selected from the group consisting of —COOH, —COO(CH₃), —COO(C₂H₅), —C(O)NH(CH₃), —C(O)NH(C₂H₅), and —C(O)-(common amino acid moiety).

In one embodiment R₁ is selected from the group consisting of methyl, —O-methyl, —OH, —OC(O)CH₃ and —COOCH₃, R₂ is selected from the group consisting of CH₂, O, and CH—CH₃, CH—C₂H₅, and R₃ is —COOH.

In one embodiment R₁ is selected from the group consisting of methyl, ethyl, —O-methyl, —O-ethyl, OH, —OC(O)CH₃, —OC(O)C₂H₅, —COOCH₃ and —COOC₂H₅, R₂ is selected from the group consisting of CH₂, O, and CH—CH₃, CH—C₂H₅, and R₃ is —COOH.

In one embodiment R₁ is selected from the group consisting of methyl and —OH, R₂ is selected from the group consisting of CH₂ and O and R₃ is selected from the group consisting of —COOH, —COO(C₁₋₃alkyl), —C(O)NH(C₁₋₃alkyl), —C(O)-(common amino acid moiety), more preferred from the group consisting of —COOH, —COO(CH₃), —COO(C₂H₅), —C(O)NH(CH₃), —C(O)NH(C₂H₅), and —C(O)-(common amino acid moiety).

The term “C₁₋₆alkyl” means a saturated linear or branched hydrocarbon group including, for example, methyl, ethyl, isopropyl, t-butyl, pentyl, hexyl, and the like. The term “C₁₋₃alkyl” means methyl, ethyl, n-propyl or isopropyl.

The term “common amino acid moiety” means the naturally occurring α-amino acids, unnatural amino acids, substituted β and γ amino acids and their enantiomers. Non-limiting examples are alanine, β-alanine, arginine, asparagine, aspartic acid, cysteine, glutamic acid, glutamine, glycine, histidine, isoleucine, leucine, lysine, methionine, phenylalanine, praline, serine, threonine, tryptophan, tyrosine, valine, 3-hydroxyproline, N-methylphenylalanine, N-methylisoleucine, norvaline, norleucine, ornithine, 2-aminobutyric acid, 2-aminoadipic acid, methionine sulfoxide, methionine sulfone, phenylglycine, o-methyltyrosine, etc.

As is well understood in this technical area, a large degree of substitution is not only tolerated, but is often advisable. Substitution is anticipated on the core structure of compounds to be used in the present invention. The term “substituents” are used to differentiate between the core structure of formula (I) and formula (II) and further chemical species that may be substituted on to the core structure. Non-limiting examples of suitable substituents may be hydrocarbon alkyl substituents, such as methyl, ethyl, propyl, t-butyl, and the like, and further substituents known in the art, such as hydroxy, alkoxy, alkylsulfonyl, halogen, cyano, nitro, amino, carboxyl, aryl, heteroaryl, cycloalkyl, common amino acids etc. It is well-known that these substituents may include further substitution, such for example, alkyl, aryl, heteroaryl etc. bearing further substituents known in the art, such as hydroxy, alkoxy, alkylsulfonyl, halogen atoms, cyano, nitro, amino, carboxyl, common amini acids etc.

The term “aryl” means a mono- or polycyclic aromatic hydrocarbon group.

The term “heteroaryl” means a monovalent aromatic cyclic radical having one to three rings, of four to eight atoms per ring, incorporating one or two heteroatoms (chosen from nitrogen, oxygen, or sulphur) within the ring.

The term “cycloalkyl” means a monovalent saturated carbocyclic radical consisting of one, two or three rings, of three to eight carbons per ring.

When the compounds of the present invention contain asymmetric carbon atoms, the pharmaceutical acceptable salts, esters, solvates and prodrugs may exist as single stereoisomers, racemates, and/or mixtures of enantiomers and/or diastereomers. All such single stereoisomers, racemates, and mixtures thereof are intended to be within the scope of the present invention.

The compound of the invention may be provided in any form suitable for the intended administration. Suitable forms include pharmaceutically acceptable salts, esters, solvates and prodrugs of the compound of Formula (I). All components must be “pharmaceutically acceptable” in the sense of being compatible with the other components of the formulation and not deleterious to the recipient thereof.

Pharmaceutically acceptable salts refer to salts of the compounds of the invention, which are considered to be acceptable for clinical and/or veterinary use. Typical pharmaceutically acceptable salts include those salts prepared by reaction of the compounds of the invention with a mineral or organic acid or an organic or inorganic base. Such salts are known as acid addition salts and base addition salts, respectively. It will be recognized that the particular counterion forming a part of any salt of this invention is not of a critical nature, so long as the salt as a whole is pharmaceutically acceptable and as long as the counterion does not contribute undesired qualities to the salt as a whole. These salts may be prepared by methods known to the skilled person.

Examples of pharmaceutically acceptable addition salts include acid addition salts formed with inorganic acids e.g. hydrochloric, hydrobromic, sulfuric, nitric, hydroiodic, metaphosphoric, or phosphoric acid; and organic acids e.g. succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, methanesulfonic or naphthalenesulfonic acid; and base addition salts formed with alkali metals and alkaline earth metals and organic bases such as N,N-dibenzylethylenediamine, chloroprocaine, choline, diethanolamine, ethylenediamine, meglumine (N-methylglucamine), lysine and procaine; and internally formed salts. Examples of suitable metal cations include sodium cation (Na+), potassium cation (K+), magnesium cation (Mg2+), calcium cation (Ca2+), and the like. (see, for example, Berge S. M. et al., “Pharmaceutical Salts,” J. of Pharma. Sci., 1977; 66:1). As used herein, the term “solvate” means a compound of the invention or a salt thereof that further includes a stoichiometric or non-stoichiometric amount of a solvent bound by non-covalent intermolecular forces. Preferred solvents are volatile, non-toxic, and/or acceptable for administration to humans in trace amounts. The solvated forms, including hydrated forms, are equivalent to unsolvated forms and are encompassed within the scope of the present invention.

As used herein, the term “prodrug” means a compound that is transformed in vivo to yield a compound of the present invention. The transformation may occur by various mechanisms, such as through hydrolysis in blood. For example, when a compound of the present invention contains a carboxylic acid functional group, a prodrug can comprise an ester formed by the replacement of the hydrogen atom of the acid group with a group including, but not limited to, groups such as for example (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N (alkoxycarbonyl)amino)ethyl having from 4 to 10 carbon atoms, 3-phthalidyl, 4 crotonolactonyl, gamma-butyrolacton-4-yl, di-N,N—(C₁-C₂)alkylamino(C₂-C₃)alkyl, carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl. The prodrug can furthermore comprise e.g. an amide formed by the replacement of the hydrogen atom of an acid group with a common amino acid moiety, non-limiting examples of common amino acids are mentioned herein above.

An even more preferred embodiment of the present invention relates to the use of a compound selected from the group consisting of isosteviol and steviol, or pharmaceutically acceptable salts or esters, solvates, or prodrugs thereof, with the proviso that the active compound is not stevioside, steviolbioside, rebaudioside A, rebaudioside B or rebaudioside C. In a preferred embodiment, the compound is isosteviol(ent-16-ketobeyeran-19-oic acid), or pharmaceutically acceptable salts or esters, solvates, or prodrugs thereof. Alternatively, the compound is steviol(ent-kaur-16-en-13-ol-19-oic acid), or pharmaceutically acceptable salts or esters, solvates, or prodrugs thereof.

As regards pharmaceutically acceptable esters of the active compound, such esters include esters formed when the carboxylic acid group has reacted with an alcohol so as to form a —COOR′ group, where R′ may represent C₁₋₆-alkyl, such as for example methyl, ethyl, n-propyl, isopropyl, n-butyl, isobutyl, sec-butyl, tert-butyl, n-pentyl, isopentyl or neopentyl. In some embodiments the C₁₋₆-alkyl may be substituted with one or more substituents. Such substituents may be selected from the group consisting of fluoro, chloro, hydroxy, methyl, ethyl, trifluoromethyl, methoxy, ethoxy, amino, methylamino, dimethylamino or —O—NO₂. Preferably, R′ represents isopropyl, n-butyl, n-butyl substituted with —O—NO₂, hydroxymethyl or 2,3-dihydroxypropyl. In other embodiments R′ may represent —(CH₂)ₙ—O—(C═O)—R¹, where n may represent an integer of 0, 1, 2, 3 or 4 and where R¹ represent methyl, ethyl, n-propyl, isopropyl, benzyl, nitrobenzyl, fluorobenzyl, chlorobenzyl, methylbenzyl or pyridinyl. When n is 0, then R¹ is preferably isopropyl, when n is 1, then R¹ is preferably methyl, benzyl, nitrobenzyl or pyridinyl. When n represent 4, then R¹ is preferably pyridinyl.

The invention further relates to a pharmaceutical composition comprising one or more compounds as defined herein, such as a mixture of steviol and isosteviol, or pharmaceutically acceptable salts or esters, solvates, or prodrugs thereof.

The compound and the pharmaceutical composition according to the invention is useful in the treatment of cognitive impairment. The terms “treatment” and “treating” as used herein refer to the management and care of a patient for the purpose of: alleviating or relieving symptoms or complications; reducing or delaying the progression of the condition, disease or disorder.

The patients to be treated according to the present invention can be of various ages. In one embodiment the patients to be treated according to the invention are over 40 years of age, such as over 50 years of age, more preferably over 60 years of age.

The beneficial effect is achieved whenever the mammal is administered an effective pharmaceutical amount of the compound according to the present invention. By the term “effective pharmaceutical amount” is meant the amount required in order to induce a beneficial effect in terms of alleviating or relieving symptoms or complications and/or reducing or delaying the progression of the cognitive impairment.

The individual in need of treatment normally receives a daily dosage of one or more of the compounds according to the invention. In the context of the present invention, the term “daily dosage” is meant to describe the daily dosage required for an average human subject having a weight of about 65 to about 70 kg.

In one embodiment of the present invention the compound is given in a daily dosage in a range of from about 0.1 g to about 20 g, such as from about 0.5 g to about 20 g, such as e.g., from about 1.0 g to about 15 g, about 1 g to about 10 g, about 1 g to about 7.5 g, about 1 g to about 5 g, about 2 g to about 5 g.

In a preferred embodiment of the present invention the compound is isosteviol, or pharmaceutical acceptable salts or esters, solvates, or prodrugs thereof, and the daily dosage is in a range of from about 0.1 g to about 15 g, such as from about 0.1 g to about 10 g, about 0.5 g to about 10 g, about 1 g to about 10 g, about 1 g to about 7.5 g, about 2 g to about 7 g, about 3 g to about 6 g, about 4 g to about 6 g, preferably from about 1 g to about 7 g, about 2 g to about 6 g, about 5 to about 6 g or about 1 g to about 5 g. Alternatively, the compound is steviol, or pharmaceutical acceptable salts or esters, solvates, or prodrugs thereof, and the daily dosage is in a range of from about 0.1 g to about 15 g, such as from about 0.1 g to about 10 g, about 0.5 g to about 10 g, about 1 g to about 10 g, about 1 g to about 7.5 g, about 2 g to about 7 g, about 3 g to about 6 g, about 4 g to about 6 g, preferably from about 1 g to about 7 g, about 2 g to about 6 g, about 5 to about 6 g or about 1 g to about 5 g.

In one embodiment the compound is isosteviol and the daily dosage is in the range of from 0.1 to 20 g, such as from 0.1 g to 0.5 g, such as from 0.5 g to 5 g, such as from 0.5 g to 2.5 g, such as from 2.5 g to 5 g, such as from 5 g to 10 g, such as from 5 g to 7.5 g, such as from 7.5 g to 10 g, such as from 10 g to 15 g, such as from 10 g to 12.5 g, such as from 12.5 g to 15 g, such as from 15 g to 20 g, such as from 15 g to 17.5 g, such as from 17.5 g to 20 g.

In one embodiment the compound is isosteviol and the daily dosage is in the range of from 0.1 g to 20 g, such as from 0.1 g to 1 g, such as from 1 g to 2 g, such as from 2 g to 3 g, such as from 3 g to 4 g, such as from 4 g to 5 g, such as from 5 g to 6 g, such as from 6 g to 7 g, such as from 7 g to 8 g, such as from 8 g to 9 g, such as from 9 g to 10 g, such as from 10 g to 11 g, such as from 11 g to 12 g, such as from 12 g to 13 g, such as from 13 g to 14 g, such as from 14 g to 15 g, such as from 15 g to 16 g, such as from 16 g to 17 g, such as from 17 g to 18 g, such as from 18 g to 19 g, such as from 19 g to 20 g.

In one embodiment the compound is isosteviol and the daily dosage is in the range of from 0.1 g to 10 g, such as from 0.1 g to 9 g, such as from 0.1 g to 8 g, such as from 0.1 g to 7 g, such as from 0.1 g to 6 g, such as from 0.1 g to 5 g, such as from 0.1 g to 4 g, such as from 0.1 g to 3 g, such as from 0.1 g to 2 g, such as from 0.1 g to 1 g.

In one embodiment the compound is isosteviol and the daily dosage is in the range of from 0.1 g to 10 g, such as from 0.5 g to 10 g, such as from 1 g to 10 g, such as from 2 g to 10 g, such as from 3 g to 10 g, such as from 4 g to 10 g, such as from 5 g to 10 g, such as from 6 g to 10 g, such as from 7 g to 10 g, such as from 8 g to 10 g.

In one embodiment the compound is isosteviol and the daily dosage is in the range of from 2 g to 15 g, such as from 2 g to 14 g, such as from 2 g to 13 g, such as from 2 g to 12 g, such as from 2 g to 11 g, such as from 2 g to 10 g, such as from 2 g to 9 g, such as from 2 g to 8 g, such as from 2 g to 7 g such as from 2 g to 6 g, such as from 6 g to 5 g.

In one embodiment the compound is isosteviol and the daily dosage is in the range of from 2 g to 15 g, such as from 3 g to 15 g, such as from 4 g to 15 g, such as from 5 g to 15 g, such as from 6 g to 15 g, such as from 7 g to 15 g, such as from 8 g to 15 g.

In one embodiment the compound is isosteviol and the daily dosage is in the range of from 0.1 g to 5 g, such as from 0.5 g to 5 g, such as from 1 g to 4 g, such as from 2 g to 3 g, such as from 0.5 g to 1 g, such as from 1 g to 1.5 g, such as from 1.5 g to 2 g, such as from 2 g to 2.5 g, such as from 2.5 g to 3 g, such as from 3 g to 3.5 g, such as from 3.5 g to 4 g, such as from 4 g to 4.5 g, such as from 4.5 g to 5 g.

In one embodiment the compound is steviol and the daily dosage is in the range of from 0.1 to 20 g, such as from 0.1 g to 0.5 g, such as from 0.5 g to 5 g, such as from 0.5 g to 2.5 g, such as from 2.5 g to 5 g, such as from 5 g to 10 g, such as from 5 g to 7.5 g, such as from 7.5 g to 10 g, such as from 10 g to 15 g, such as from 10 g to 12.5 g, such as from 12.5 g to 15 g, such as from 15 g to 20 g, such as from 15 g to 17.5 g, such as from 17.5 g to 20 g.

In one embodiment the compound is steviol and the daily dosage is in the range of from 0.1 g to 20 g, such as from 0.1 g to 1 g, such as from 1 g to 2 g, such as from 2 g to 3 g, such as from 3 g to 4 g, such as from 4 g to 5 g, such as from 5 g to 6 g, such as from 6 g to 7 g, such as from 7 g to 8 g, such as from 8 g to 9 g, such as from 9 g to 10 g, such as from 10 g to 11 g, such as from 11 g to 12 g, such as from 12 g to 13 g, such as from 13 g to 14 g, such as from 14 g to 15 g, such as from 15 g to 16 g, such as from 16 g to 17 g, such as from 17 g to 18 g, such as from 18 g to 19 g, such as from 19 g to 20 g.

In one embodiment the compound is steviol and the daily dosage is in the range of from 0.1 g to 10 g, such as from 0.1 g to 9 g, such as from 0.1 g to 8 g, such as from 0.1 g to 7 g, such as from 0.1 g to 6 g, such as from 0.1 g to 5 g, such as from 0.1 g to 4 g, such as from 0.1 g to 3 g, such as from 0.1 g to 2 g, such as from 0.1 g to 1 g.

In one embodiment the compound is steviol and the daily dosage is in the range of from 0.1 g to 10 g, such as from 0.5 g to 10 g, such as from 1 g to 10 g, such as from 2 g to 10 g, such as from 3 g to 10 g, such as from 4 g to 10 g, such as from 5 g to 10 g, such as from 6 g to 10 g, such as from 7 g to 10 g, such as from 8 g to 10 g.

In one embodiment the compound is steviol and the daily dosage is in the range of from 2 g to 15 g, such as from 2 g to 14 g, such as from 2 g to 13 g, such as from 2 g to 12 g, such as from 2 g to 11 g, such as from 2 g to 10 g, such as from 2 g to 9 g, such as from 2 g to 8 g, such as from 2 g to 7 g such as from 2 g to 6 g, such as from 6 g to 5 g.

In one embodiment the compound is steviol and the daily dosage is in the range of from 2 g to 15 g, such as from 3 g to 15 g, such as from 4 g to 15 g, such as from 5 g to 15 g, such as from 6 g to 15 g, such as from 7 g to 15 g, such as from 8 g to 15 g.

In one embodiment the compound is steviol and the daily dosage is in the range of from 0.1 g to 5 g, such as from 0.5 g to 5 g, such as from 1 g to 4 g, such as from 2 g to 3 g, such as from 0.5 g to 1 g, such as from 1 g to 1.5 g, such as from 1.5 g to 2 g, such as from 2 g to 2.5 g, such as from 2.5 g to 3 g, such as from 3 g to 3.5 g, such as from 3.5 g to 4 g, such as from 4 g to 4.5 g, such as from 4.5 g to 5 g.

The daily dosage may optionally be administered as a single dose or be divided in two or more doses, such as e.g. two, three, or four, for administration at different times during the day. The physician will in any event determine the actual dosage which will be most suitable for any particular patient and it will vary with the age, weight and response of the particular patient. The above dosages are, of course only exemplary of the average case and there may be instances where higher or lower doses are merited and such are within the scope of the invention.

Another way of expressing the daily dosage level in accordance with the present invention is as mg/kg. Accordingly, for administration to human patients the daily dosage levels of the compounds in accordance with the present invention, or pharmaceutically acceptable salts or esters, solvates, or prodrugs thereof, will be in a range from about 10 to about 350 mg/kg, preferably from about 20 to about 100 mg/kg, and more preferably from about 20 to about 50 mg/kg. Alternatively, the daily dosage is in the range from about 1 to 20 mg/kg, such as from 2 to 15 mg/kg, such as from 5 to 10 mg/kg.

In a preferred embodiment the dosages are based on the weight of the core of formula (I), so that for example the compound is given in a daily dosage in a range of from about 0.5 g to about 20 g of the core, such as e.g., from about 1.0 g to about 15 g of the core, about 1 g to about 10 g of the core, about 1 g to about 7.5 g of the core, about 1 g to about 5 g of the core, about 2 g to about 5 g of the core.

The compounds for use in accordance with the present invention may be administered alone, or as part of a combination therapy. If a combination of active agents is administered, then it may be administered simultaneously, separately or sequentially. Depending on the disease and the state of the disease to be treated, it may be relevant to include one or more additional active compound in the medicament.

The main routes of administration are oral and parenteral in order to introduce the agent into the blood stream to ultimately target the sites of desired action.

Appropriate dosage forms for such administration may be prepared by conventional techniques.

Oral administration is normally for enteral drug delivery, wherein the agent is delivered through the enteral mucosa.

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration, subcutaneous administration. Furthermore, parenteral administration includes inhalations and topical administration.

Accordingly, the agent may be administered topically to cross any mucosal membrane of an animal to which the biologically active substance is to be given, e.g. in the nose, vagina, eye, mouth, genital tract, lungs, gastrointestinal tract, or rectum, the mucosa of the nose, or mouth, and accordingly, parenteral administration may also include buccal, sublingual, nasal, rectal, vaginal and intraperitoneal administration as well as pulmonal and bronchial administration by inhalation or installation. Also, the agent may be administered topically to cross the skin.

Preferably the administration route is the oral route.

### Formulations

For use in the present invention the compounds may be administered alone, but will generally be administered in admixture with suitable pharmaceutical excipients, diluents or carriers selected with regard to the intended route of administration and standard pharmaceutical practice.

For example, the compounds to be used in accordance with the invention can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via fast dispersing or fast dissolving dosage forms.

Tablets may contain excipients such as microcrystalline cellulose, lactose, sodium citrate, calcium carbonate, dibasic calcium phosphate, glycine, and starch (preferably corn, potato or tapioca starch), disintegrants such as sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatine and acacia. Additionally, lubricating agents such as magnesium stearate, stearic acid, glyceryl behenate and talc may be included.

Solid compositions of a similar type may also be employed as fillers in gelatine capsules. Preferred excipients in this regard include lactose, starch, a cellulose, milk sugar or high molecular weight polyethylene glycols.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating. Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatine, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug compound used i.e. where the drug compound is insoluble a fast dispersing dosage form can be prepared and where the drug compound is soluble a fast dissolving dosage form can be prepared.

In general a tablet formulation could typically contain between about 0.5 g to about 5 g of a compound for use in accordance with the present invention (or a salt, ester, solvate or prodrug thereof) whilst tablet fill weights may for example range from 50 mg to 3000 mg. An example formulation for a tablet is illustrated here:

| Ingredient | % w/w |
|---|---|
| Steviol, Isosteviol, or salts or esters, | 100.000* |
| solvates, or prodrugs thereof | |
| Lactose | 64.125 |
| Starch | 21.375 |
| Croscarmellose Sodium | 3.000 |
| Magnesium Stearate | 1.500 |

*This quantity is typically adjusted in accordance with the desired dosage.

Another example formulation is illustrated here.

| Ingredient | Amount, mg |
|---|---|
| Isosteviol | 1000* |
| Starch | 259 |
| Lactose | 259 |
| Magnesium stearate | 3.3 |
| Talc | 29.7 |

*This quantity is typically adjusted in accordance with the desired dosage

The above example formulations may further contain e.g. colour, flavour or a coating in order to disguise an unpleasant taste.

Yet another example may be a capsule formulation:

| Ingredient | Amount, mg |
|---|---|
| Isosteviol | 1000* |
| Starch | 10-1000 |
| Gelatine capsule | |

*This quantity is typically adjusted in accordance with the desired dosage

For aqueous suspensions and/or elixirs, the compounds of the invention, or the pharmaceutically acceptable salts or esters, solvates, or prodrugs thereof, may be combined with various sweetening or flavouring agents, colouring matter or dyes, with emulsifying and/or suspending agents and with diluents such as water, ethanol, propylene glycol and glycerine, and combinations thereof.

The compounds for use in accordance with the invention can also be administered parenterally, as discussed above, or they may be administered by infusion techniques. For such parenteral administration medicaments are best used in the form of a sterile aqueous solution which may contain other compounds, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

The compounds for use in accordance with the invention can also be administered intranasally or by inhalation and are conveniently delivered in the form of a dry powder inhaler or an aerosol spray presentation from a pressurised container, pump, spray or nebulizer with the use of a suitable propellant, e.g. dichlorodifluoromethane, trichlorofluoromethane, dichlorotetra-fluoro-ethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatine) for use in an inhaler or insufflator may be formulated to contain a powder mix of compound for use in accordance with the invention and a suitable powder base such as lactose or starch. The compounds for use in accordance with the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds for use in accordance with the invention can be administered by the rectal or topical route. This may be in the form of a suppository, or by topical application in the form of a gel, hydrogel, lotion, solution, cream, ointment, dusting powder or skin patch. For application topically to the skin, the compounds can be formulated as a suitable ointment containing the active compound suspended or dissolved in, for example, a mixture with one or more of the following: mineral oil, liquid petrolatum, white petrolatum, propylene glycol, polyoxyethylene, polyoxypropylene compound, emulsifying wax and water. Alternatively, the compounds can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters, wax, cetearyl alcohol, 2-octyldodecanol, benzyl alcohol and water.

The compounds for use in accordance with of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

In addition to the above described formulations, medicaments containing a compound for use in accordance with the present invention may furthermore be prepared by conventional techniques, e.g. as described in Remington: The Science and Practice of Pharmacy 1995, edited by E. W. Martin, Mack Publishing Company, 19th edition, Easton, Pa.

### EXAMPLES

### Examination of the Effect of Isosteviol on Cognitive Function in Elderly Patients with Alzheimer's Disease

Healthy volunteers and a subject groups comprising elderly patients with Alzheimers disease and/or mild cognitive impairment receive relevant dosages of a Isosteviol as described herein as a single dose. The blood levels of isosteviol is monitored and the volunteers/patients are tested for changes in cognitive function with a variety of psychometric tests which include the tests mentioned in “A Neuropsycological Test Battery for use in Alzheimer Disease Clinical Trials”, ARCH NEUROL, September 2007, vol. 64, no. 9, pp. 1323-1329. Then the effect is evaluated. The volunteers/patients are also subjected to a quantitative assessment of the changes produced in the awake electroencephalogram (EEG) particularly expressed in terms of the ratio of the alpha wave power to that of the lower frequency waves (delta and theta).

### Determination of Dose-Response

The abovementioned experiments may also be performed on healthy volunteers and a subject group comprising elderly patients with Alzheimer's disease and mild cognitive impairment and the results may then be used to determine the relationship between the change in blood isosteviol level and the change in cognitive function and the quantitative EEG data, i.e. determination of a dose-response curve.

### Effect of Isosteviol in a Human Model of Alzheimer's Disease

Alzheimer-like changes in both the EEG and cognitive function measures may be provoked in healthy volunteers by administration of scopolamine. This is a well-established experimental model of Alzheimer's disease, which is disclosed in “The scopolamine model of dementia: determination of central cholinomimetic effects of physostigmine on cognition and biochemical markers in man”, J. Psychopharmacol., March 1988, vol. 2, no. 2, pp. 67-79. The experimental model has been widely used for the evaluation of drugs with anti-alzheimer potential.

### Evaluation of the Ability of Isosteviol to Attenuate Scopolamine Induced Learning and Memory Deficit in Rats in the Morris Water Maze Test

The Morris water maze test is a classical cognitive test for evaluation of spatial learning and memory in rodents. Scopolamine induced learning and memory deficit in Morris water maze test is the most common memory deficit model for memory enhancing screenings.

60 Sprague-Dawley male rats were used in the present study. The animals were housed in cages in groups of four rats per cage. All rats were fed with food and water ad libitum.

The testing apparatus can be described as follows. A circular pool was filled with water (24±1° C.) to the depth of 30 cm. A circular escape platform was placed in the fourth quadrant of the pool submerged 2 cm beneath the surface of the water. Four start positions were at the perimeter of the pool: the first one in the first quadrant, the second one in the second quadrant, the third one in the third quadrant and the last one in the fourth quadrant. The rats were trained to escape from water by swimming to a hidden platform whose location was only identified using visual cues attached to the curtain around the pool. Visual cues had different colors and dimensions and were kept constant during the whole experiment.

All rats were trained in the swimming pool for 60 seconds without the platform. Each rat was trained to learn the position of submerged platform for consecutive 5 days. The training procedure included 4 trials per day with 4 different and randomized starting positions. In each trial, the time of rat to reach the platform (escape latency) was recorded, and then the rat was allowed to stay on the platform for 15 seconds. If a rat did not reach the platform within 60 seconds it was guided to the platform and allowed to stay on the platform for 30 seconds.

The experimental group setting is described in Table 3. Scopolamine was administered to induce learning and memory deficit, and saline was used as control vehicle. Donepezil and stevioside were administered as positive control compounds, whereas isosteviol was administered as the test compound.

TABLE 3
Experimental group setting.

| | | Dose | Route | | Dose | Route | |
|---|---|---|---|---|---|---|---|
| | 1^{st} | (mg/ | of | 2^{nd} | (mg/ | of | |
| Group | treatment | kg) | admin. | treatment | kg) | admin. | N |
| 1 | 0.5% | — | po | Saline | — | ip | 10 |
| | MC* | | | | | | |
| 2 | 0.5% | — | po | Scopolamine | 0.5 | Ip | 10 |
| | MC* | | | | | | |
| 3 | Isosteviol | 80 | po | Scopolamine | 0.5 | Ip | 10 |
| 4 | Isosteviol | 250 | po | Scopolamine | 0.5 | Ip | 10 |
| 5 | Stevioside | 250 | po | Scopolamine | 0.5 | Ip | 10 |
| 6 | Donepezil | 0.1 | Ip | Scopolamine | 0.5 | ip | 10 |

*MC = methyl cellulose

For practical reasons, the study was divided into two sessions (Table 4). Each session included 6 groups, and each group included 5 rats. Each session took 10 days to complete.

TABLE 4

| | |
|---|---|
| Session 1 | Session 2 |

| Group | N | Group | N |
|---|---|---|---|
| Saline | 5 | Saline | 5 |
| Scopolamine 0.5 mg/kg | 5 | Scopolamine 0.5 mg/kg | 5 |
| Isosteviol 80 mg/kg | 5 | Isosteviol 80 mg/kg | 5 |
| Isosteviol 250 mg/kg | 5 | Isosteviol 250 mg/kg | 5 |
| Stevioside 250 mg/kg | 5 | Stevioside 250 mg/kg | 5 |
| Donepezil 0.1 mg/kg | 5 | Donepezil 0.1 mg/kg | 5 |

Scopolamine was dosed 30 minutes before acquisition trial from day 1 to day 5. Isosteviol, stevioside and Donepezil hydrochloride were administered 30 minutes before administration of Scopolamine.

On day 6, each rat was placed in the swimming pool with no platform to locate the platform site for 60 seconds. Each rat was administered saline 30 minutes before probe trial.

Any Maze software was used for video tracking and data collection during the whole acquisition trial and probe trial. In acquisition trial, escape latency was recorded. In probe trial, time spent in target and swimming speed was recorded. Data were analyzed and graphed by Graph Pad Prism 5.

The results are shown in Table 1 and 2. Table 1 shows the latency (in seconds) to escape in acquisition trial on day 1, 2, 3, 4, and 5, respectively, for each group. Table 2 shows the time spent in target quadrant in probe trial on day 6.

TABLE 1
Latency to escape in acquisition trial in session 1 and 2 Tabel 3?

| | | Saline | Scopol. | Isosteviol | Isosteviol | Stevioside | Donepezil |
|---|---|---|---|---|---|---|---|
| | Group | (s) | 0.5 mg/kg (s) | 80 mg/kg (s) | 250 mg/kg (s) | 250 mg/kg (s) | 0.1 mg/kg (s) |
| Day 1 | Ses. 1 | 44.44 ± 4.82 | 45.92 ± 5.82 | 46.46 ± 4.50 | 46.16 ± 2.48 | 44.98 ± 5.53 | 48.52 ± 5.50 |
| | Ses. 2 | 44.62 ± 4.09 | 46.30 ± 2.47 | 45.20 ± 6.40 | 48.46 ± 4.49 | 46.48 ± 3.66 | 45.44 ± 5.40 |
| Day 2 | Ses. 1 | 43.50 ± 4.07 | 46.38 ± 2.26 | 45.24 ± 7.87 | 43.94 ± 5.18 | 45.78 ± 6.95 | 44.50 ± 2.48 |
| | Ses. 2 | 41.72 ± 2.71 | 44.08 ± 4.81 | 41.88 ± 3.19 | 50.76 ± 3.90 | 40.44 ± 3.63 | 43.18 ± 7.85 |
| Day 3 | Ses. 1 | 30.60 ± 6.96 | 42.96 ± 8.49 | 40.42 ± 5.48 | 48.62 ± 5.64 | 44.80 ± 5.46 | 35.92 ± 8.22 |
| | Ses. 2 | 29.46 ± 3.08 | 45.28 ± 4.39 | 36.04 ± 5.50 | 40.42 ± 4.57 | 39.46 ± 9.62 | 37.06 ± 4.26 |
| Day 4 | Ses. 1 | 22.88 ± 5.43 | 42.60 ± 7.90 | 37.84 ± 4.36 | 44.36 ± 6.54 | 41.74 ± 6.39 | 36.04 ± 4.78 |
| | Ses. 2 | 26.56 ± 5.54 | 42.10 ± 3.19 | 38.74 ± 5.25 | 41.58 ± 6.33 | 35.78 ± 4.95 | 34.20 ± 6.89 |
| Day 5 | Ses. 1 | 19.20 ± 3.41 | 38.20 ± 8.79 | 27.14 ± 4.30 | 41.44 ± 7.24 | 30.86 ± 6.57 | 26.86 ± 3.09 |
| | Ses. 2 | 20.82 ± 2.80 | 36.50 ± 4.93 | 33.38 ± 6.04 | 35.24 ± 7.74 | 31.08 ± 3.33 | 30.50 ± 5.78 |

TABLE 2
Time spent in target quadrant in probe
trial in session 1 and 2 Tabel 4?

| Group | Session 1(s) | Session (2) |
|---|---|---|
| Saline | 24.08 ± 0.83 | 27.80 ± 2.59 |
| Scopolamine 0.5 mg/kg | 15.70 ± 1.42 | 12.30 ± 1.24 |
| Isosteviol 80 mg/kg | 19.80 ± 1.19 | 17.28 ± 1.11 |
| Isosteviol 250 mg/kg | 16.00 ± 2.37 | 16.50 ± 1.61 |
| Stevioside 250 mg/kg | 20.78 ± 3.75 | 21.10 ± 2.16 |
| Donepezil 0.1 mg/kg | 18.84 ± 3.22 | 19.40 ± 1.09 |

The results of this investigation reveals that on day 1 of the acquisition trials, there was no significant difference in the average time for all test group rats to locate the submerged platform (vehicle, 44.53±2.98 s; Scopolamine, 46.11±2.98 s; Isosteviol 80 mg/kg, 45.83±3.70 s; Isosteviol 250 mg/kg, 47.31±2.45 s; Stevioside, 45.73±3.14 s; Donepezil, 46.98±3.67 s).

In both sessions on day 5 in acquisition trials, vehicle control rats showed a significant decrease in latency to escape, as compared with that on day 1, indicating a successful learning and memory process and capability shown in normal rats in the model.

On day 5, scopolamine at the dose of 0.5 mg/kg, as a positive model creation group, significantly increased in latency to the submerged platform in acquisition trials and reduced time to locate in target quadrant in probe trials. The positive results of scopolamine induced deficits in both acquisition and probe trials indicated the successful model creation of scopolamine induced learning and memory deficit in the Morris water maze model with a significant window for the deficit recovery.

Donepezil, as a positive control at the dose of 0.1 mg/kg significantly attenuated learning and memory deficit induced by scopolamine, as indicated by comparing the time to locate in target quadrant in probe trials, between donepezil+scopolamine and scopolamine only groups. It suggested the model system be well developed for the positive control to show efficacy.

Stevioside as a target validation positive control at the dose of 250 mg/kg also significantly reversed learning and memory deficit induced by scopolamine in probe trials, suggesting a valid target somehow for learning and memory deficit recovery.

Isosteviol at the dose of 80 mg/kg significantly attenuated learning and memory deficit in probe trials, but not at the dose of 250 mg/kg. There were no visible behavioral abnormalities that in the premise of the right model and correct conclusions applied for the study equal or smaller doses than 80 mg/kg of isosteviol show a dose dependent efficacy, whereas, equal or greater than 250 mg/kg of isosteviol show less or no efficacy in the same Morris water maze model.

There was no significant difference found among test groups regarding mean speed in probe trials, indicating that test compounds do not affect locomotor functions.

In comparison of the results of acquisition trials and probe trials there were more statistical significances seen in probe trials among groups, suggesting the necessity of the probe trial in the Morris water maze model and that the probe trial be more sensitive to detect the differences in learning and memory functions in the study.

1. A pharmaceutically active compound comprising the core structure of formula (I) 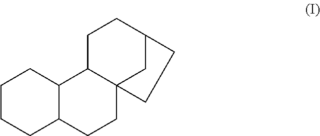 [SMILES: C1CCC2C(C1)CCC13CCC(CCC21)C3]
or a pharmaceutically acceptable salt, ester, solvate, or prodrug of said active compound,
with the proviso that the active compound is not stevioside, steviolbioside, rebaudioside A, rebaudioside B or rebaudioside C.

2. The compound according to claim 1, wherein the core structure of formula (I), is a core structure of formula (II) 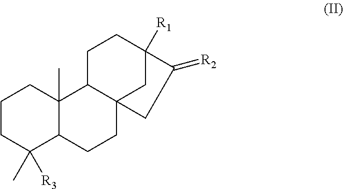
wherein
R₁ is selected from the group consisting of —C₁₋₆alkyl, —O—C₁₋₆alkyl, —OH, and —OC(O)(C₁₋₆alkyl), —COO(C₁₋₆alkyl);
R₂ is selected from the group consisting of CH₂, O, and CH(C₁₋₆alkyl); and
R₃ is selected from the group consisting of —COOH, —COO(C₁₋₆alkyl), —C(O)NH(C₁₋₆alkyl), and —C(O)-(common amino acid moiety).

3. The compound according to claim 1, wherein the compound is selected from the group consisting of isosteviol and steviol.

4. The compound according to claim 1, wherein the compound is selected from the group consisting of isosteviol, steviol, and pharmaceutically acceptable salts, esters, solvates, and prodrugs thereof.

5. The compound according to claim 1, wherein the compound is isosteviol, or a pharmaceutically acceptable salt, ester, solvate, or prodrug thereof.

6. The compound according to claim 1, wherein the compound is steviol, or a pharmaceutically acceptable salt, ester, solvate, or prodrug thereof.

7. The compound according to claim 1, wherein the compound is given in a daily dosage in a range of from about 0.1 g to about 20 g.

8. The compound according to claim 1, wherein the compound is steviol and the daily dosage is in a range of from about 0.1 g to about 15 g.

9. The compound according to claim 1, wherein the compound is isosteviol and daily dosage is in a range of from about 0.1 g to about 15 g.

10. The compound according to claim 1, wherein the daily dosage is in a range from about 1 to 20 mg/kg.

11. The compound according to claim 1, wherein steviol or isosteviol is isolated from a plant source.

12. A composition comprising the compound of claim 1.

13. The composition according to claim 12, further comprising one or more additional active compound(s).

14. The composition according to claim 13, wherein the compound is a mixture of steviol and isosteviol, or pharmaceutically acceptable salts, esters, solvates, or prodrugs thereof.

15. A method of treating cognitive impairment in a mammalian subject in need thereof, said method comprising administering a therapeutically effective amount of a compound according to claim 1 to the mammalian subject.

16. The method according to claim 15, where the cognitive impairment is selected from the group consisting of Alzheimer's, Dementia with Lewy bodies, Parkinson's disease, dementia and cognitive deficiencies caused by traumas.

17. The method according to claim 15, where the mammalian subject is selected from the group consisting of human beings, dogs, cats and horses.

18. The method according to claim 15, where the therapeutically effective amount equals a daily dosage in a range of from about 0.1 g to about 15 g.
